(19) United States
(12) Patent Application Publication
(10) Pub. No.: US 2022/0168274 A1
(43) Pub. Date: Jun. 2, 2022

(54) Parenteral Formulation

(71) Applicant: Small Pharma Ltd., London (GB)

(72) Inventors: Peter Rands, London (GB); Marie Claire Layzell, London (GB)

(73) Assignee: Small Pharma Ltd., London (GB)

(21) Appl. No.: 17/108,679

(22) Filed: Dec. 1, 2020

(51) Int. Cl. A61K 31/4045; A61K 47/10; C07B 59/00; A61K 9/00; A61J 1/05

(52) U.S. Cl. CPC A61K 31/4045; A61K 47/10; A61J 1/05; A61K 9/007; C07B 59/002

(57) ABSTRACT

The present invention relates to parenteral formulations, and kits and methods suitable for the preparation of such parenteral formulations. The parenteral formulations comprise a freebase of a deuterium-substituted dimethyltryptamine optionally substituted at position 4 or 5 with acetoxy or methoxy. The parenteral formulations also comprise a biocompatible excipient. Such formulations are suitable for inhalation and have uses in the treatment of psychiatric or neurological disorders. They are metabolised more slowly than their protio analogues, allowing for a longer lasting therapeutic effect.

### FIELD

The present invention relates to parenteral formulations (i.e. formulations suitable for parenteral administration), and kits and methods suitable for the preparation of such formulations. The parenteral formulations comprise a freebase of a deuterium-substituted dimethyltryptamine optionally substituted at position 4 or 5 with acetoxy or methoxy. The parenteral formulations also comprise a biocompatible excipient. Such formulations are suitable for inhalation and have uses in the treatment of psychiatric or neurological disorders. They are metabolised more slowly than their protio analogues, allowing for a longer lasting therapeutic effect.

### BACKGROUND

Classical psychedelics have shown preclinical and clinical promise in treating psychiatric disorders (Carhart-Harris and Goodwin, Neuropsychopharmacology 42, 2105-2113 (2017)).

N,N-dimethyltryptamine (DMT) is understood to hold therapeutic value as a short-acting psychedelic. A review of research into the biosynthesis and metabolism of DMT in the brain and peripheral tissues, methods and results for DMT detection in body fluids and the brain, new sites of action for DMT, and new data regarding the possible physiological and therapeutic roles of DMT is provided by S. A. Barker in Front. Neurosci., 12, 536, 1-17 (2018). In this review, DMT is described as having a possible therapeutic role in the treatment of depression, obsessive-compulsive disorder, and substance abuse disorders.

5-methoxy-N,N-dimethyltryptamine (5-MeO-DMT) is an endogenous tryptamine found in human blood, urine, and spinal fluid (S. A. Barker, E. H. McIlhenny and R. Strassman, Drug Test. Anal., 2012, 4, 7-8, 617-635; F. Benington, R. D. Morin and L. C. Clark, J. Med. Sci., 1965, 2, 397-403; F. Franzen, and H. Gross, Nature, 206, 1052; R. B. Guchhait., J. Neurochem., 1976, 26, 1, 187-190), and has been shown to exhibit protective and therapeutically relevant effects. Anti-depressant properties have been shown in rodents administered 5-MeO-DMT (M. S. Riga et al., Neuropharmacology, 2017, 113, A, 148-155). In addition, a high number of users of 5-MeO-DMT, having ingested it in different forms, reported therapeutic effects attributed to its use, including improved post-traumatic stress disorder, depression and anxiety (A. K. Davis et al., J. Psychopharmacol., 2018, 32, 7, 779-792). 5-MeO-DMT has also exhibited the potential to treat substance abuse disorders (V. Dakic et al., Sci. Rep., 2017, 7, 12863).

Recently, the website vice.com carried out an interview with an anonymous chemist making and selling DMT vape pens (see https://www.vice.com/en/article/akzgbz/i-sell-dmt-vape-pens-so-people-can-break-through-at-their-own-speed) as available in November 2020). In the interview, the chemist describes the benefits of vaping DMT as “the ability to control how fast and how deep you want to go with each puff . . . You can still “break through” [the term for a full-blown DMT experience that involves extreme, visionary, psychedelic states] with enough hits. Or you can just have a light eyes-open, colourful trip that lasts around ten minutes. A mini-dose with gentle effects”.

In the same interview, the problem of increasing the length of the “trip” experienced by the user is discussed. The chemist's approach in increasing “trip” length is to add some Banisteriopsis Caapi (an Amazonian jungle vine) extract to the formulation used. However, it is mentioned by the chemist that the resultant formulation can cause negative reactions for people on certain medications and would only extend the trip by five minutes.

In light of the therapeutic potential of DMT and substituted dimethyltryptamines, there remains a need in the art for formulations, in particular parenteral formulations, comprising such compounds with improved availability, extended and/or modified pharmacokinetics, in particular for the development of clinically applicable psychedelic drug substances to assist psychotherapy. The present invention addresses this need.

### SUMMARY

The present invention relates to formulations suitable for parenteral administration, comprising a freebase of a deuterium-substituted DMT, which is optionally substituted at position 4 or 5 with acetoxy or methoxy, and a biocompatible excipient. Such formulations are suitable for inhalation and have uses in the treatment of psychiatric or neurological disorders. As described above, there remains a need in the art for formulations comprising DMT and substituted DMTs with improved parenteral (including inhalable) availability. The inventors have found that formulations comprising a freebase of a deuterium-substituted DMT optionally substituted at position 4 or 5 with acetoxy or methoxy are metabolised more slowly than formulations comprising protio analogues of these compounds, allowing for a longer lasting therapeutic effect.

The inventors have also found that the deuterium-substituted DMTs are more soluble in formulations suitable for parenteral administration when the compounds are in the form of a freebase rather than a salt.

Accordingly, viewed from a first aspect, the invention provides a parenteral formulation comprising a freebase of a deuterium-substituted DMT optionally substituted at position 4 or 5 with acetoxy or methoxy, and a biocompatible excipient.

Viewed from a second aspect, the invention provides a kit suitable for preparing a parenteral formulation of the first aspect, said kit comprising a freebase of a deuterium-substituted DMT optionally substituted at position 4 or 5 with acetoxy or methoxy, and a biocompatible excipient, which is separate to the freebase.

Viewed from a third aspect, the invention provides a method of preparing a parenteral formulation of the first aspect, comprising contacting a freebase of a deuterium-substituted DMT optionally substituted at position 4 or 5 with acetoxy or methoxy, with a biocompatible excipient.

Parenteral formulations, for example those suitable for inhalation, comprising a freebase of a DMT optionally substituted with deuterium and optionally substituted at position 4 or 5 with acetoxy or methoxy, and a biocompatible excipient, have uses in the treatment of psychiatric or neurological disorders. Accordingly, viewed from a fourth aspect, the invention provides a parenteral formulation of the first aspect, or a kit of the second aspect for use in therapy.

Viewed from a fifth aspect, the invention provides a parenteral formulation of the first aspect, or a kit of the second aspect for use in a method of treating a psychiatric or neurological disorder in a patient.

Viewed from a sixth aspect, the invention provides a method of treating a psychiatric or neurological disorder comprising pulmonary administration to a patient in need thereof of a parenteral formulation of the first aspect.

Further aspects and embodiments of the present invention will be evident from the discussion that follows below.

### BRIEF DESCRIPTION OF THE FIGURES

FIG. 1: plots calculated in vitro half-life for DMT and 6 deuterated-containing compositions described in the Examples section, below. A) Linear regression analysis. The r² value for half-life is 0.754; where the slope was found to be significantly different to zero, p=0.01. B) Half-life of deuterated analogues as a percent change from (undeuterated) DMT (dashed line).

FIG. 2: in vitro intrinsic clearance for DMT and 6 deuterium-containing compositions described in the Examples section, below. A) Linear regression analysis. The r² value for intrinsic clearance is 0.7648; where the slope was found to be significantly different to zero, p=0.01. B) Intrinsic clearance of deuterated analogues as a percent change from (undeuterated) DMT (dashed line).

### DETAILED DESCRIPTION

Throughout this specification, one or more aspects of the invention may be combined with one or more features described in the specification to define distinct embodiments of the invention.

In the discussion that follows, reference is made to a number of terms, which are to be understood to have the meanings provided below, unless a context expressly indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 1996, 68, 2287-2311). For the avoidance of doubt, if a rule of the IUPAC organisation is contrary to a definition provided herein, the definition herein is to prevail.

References herein to a singular of a noun encompass the plural of the noun, and vice-versa, unless the context implies otherwise. For example, “a biocompatible excipient” encompasses one or more biological excipients.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The term “comprising” includes within its ambit the term “consisting”.

The term “consisting” or variants thereof is to be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if a concentration of freebase is specified to be about 1 mg/mL to about 1000 mg/mL, concentrations of 0.95 mg/mL to 1050 mg/mL are included.

The formulation defined in the first aspect is useful in therapy and may be administered to a patient in need thereof. As used herein, the term ‘patient’ preferably refers to a mammal. Typically the mammal is a human, but may also refer to a domestic mammal. The term does not encompass laboratory mammals.

The terms “treatment” and “therapy” define the therapeutic treatment of a patient, in order to reduce or halt the rate of progression of a disorder, or to ameliorate or cure the disorder. Prophylaxis of a disorder as a result of treatment or therapy is also included. References to prophylaxis are intended herein not to require complete prevention of a disorder: its development may instead be hindered through treatment or therapy in accordance with the invention. Typically, treatment or therapy is not prophylactic, and the formulations are administered to a patient having a diagnosed or suspected disorder.

Psychedelic-assisted psychotherapy means the treatment of a mental disorder by psychological means, which are enhanced by one or more protocols in which a patient is subjected to a psychedelic experience. A psychedelic experience is characterized by the striking perception of aspects of one's mind previously unknown, and may include one or more changes of perception with respect to hallucinations, synesthesia, altered states of awareness or focused consciousness, variation in thought patterns, trance or hypnotic states, and mystical states.

As is understood in the art, psychocognitive, psychiatric or neurological disorders are disorders which may be associated with one or more cognitive impairment. As used herein, the term ‘psychiatric disorder’ is a clinically significant behavioural or psychological syndrome or pattern that occurs in an individual and that is associated with present distress (e.g., a painful symptom) or disability (i.e., impairment in one or more important areas of functioning) or with a significantly increased risk of suffering death, pain, disability, or an important loss of freedom.

Diagnostic criteria for psychiatric or neurological disorders referred to herein are provided in the Diagnostic and Statistical Manual of Mental Disorders, Fifth Edition, (DSM-5).

As used herein the term ‘obsessive-compulsive disorder’ (OCD) is defined by the presence of either obsessions or compulsions, but commonly both. The symptoms can cause significant functional impairment and/or distress. An obsession is defined as an unwanted intrusive thought, image or urge that repeatedly enters the person's mind. Compulsions are repetitive behaviours or mental acts that the person feels driven to perform. Typically, OCD manifests as one or more obsessions, which drive adoption of a compulsion. For example, an obsession with germs may drive a compulsion to clean or an obsession with food may drive a compulsion to overeat, eat too little or throw up after eating (i.e. an obsession with food may manifest itself as an eating disorder). A compulsion can either be overt and observable by others, such as checking that a door is locked, or a covert mental act that cannot be observed, such as repeating a certain phrase in one's mind.

The term “eating disorder” includes anorexia nervosa, bulimia and binge eating disorder (BED). The symptoms of anorexia nervosa include eating too little and/or exercising too much in order to keep weight as low as possible. The symptoms of bulimia include eating a lot of food in a very short amount of time (i.e. binging) and then being deliberately sick, using laxatives, eating too little and/or exercising too much to prevent weight gain. The symptoms of BED include regularly eating large portions of food until uncomfortably full, and consequently feeling upset or guilty.

As used herein the term ‘depressive disorder’ includes major depressive disorder, persistent depressive disorder, bipolar disorder, bipolar depression, and depression in terminally ill patients.

As used herein the term ‘major depressive disorder’ (MDD, also referred to as major depression or clinical depression) is defined as the presence of five or more of the following symptoms over a period of two-weeks or more (also referred to herein as a ‘major depressive episode’), most of the day, nearly every day:
depressed mood, such as feeling sad, empty or tearful (in children and teens, depressed mood can appear as constant irritability);
significantly reduced interest or feeling no pleasure in all or most activities;
significant weight loss when not dieting, weight gain, or decrease or increase in appetite (in children, failure to gain weight as expected);
insomnia or increased desire to sleep;
either restlessness or slowed behaviour that can be observed by others;
fatigue or loss of energy;
feelings of worthlessness, or excessive or inappropriate guilt;
trouble making decisions, or trouble thinking or concentrating;
recurrent thoughts of death or suicide, or a suicide attempt.

At least one of the symptoms must be either a depressed mood or a loss of interest or pleasure.

Persistent depressive disorder, also known as dysthymia, is defined as a patient exhibiting the following two features:
A. has depressed mood for most the time almost every day for at least two years. Children and adolescents may have irritable mood, and the time frame is at least one year.
B. While depressed, a person experiences at least two of the following symptoms:
   Either overeating or lack of appetite.
   Sleeping too much or having difficulty sleeping.
   Fatigue, lack of energy.
   Poor self-esteem.
   Difficulty with concentration or decision-making.

As used herein the term ‘treatment resistant major depressive disorder’ describes MDD that fails to achieve an adequate response to an adequate treatment with standard of care therapy.

As used herein, ‘bipolar disorder’, also known as manic-depressive illness, is a disorder that causes unusual shifts in mood, energy, activity levels, and the ability to carry out day-to-day tasks.

There are two defined sub-categories of bipolar disorder; all of them involve clear changes in mood, energy, and activity levels. These moods range from periods of extremely “up,” elated, and energised behaviour (known as manic episodes, and defined further below) to very sad, “down,” or hopeless periods (known as depressive episodes). Less severe manic periods are known as hypomanic episodes.

Bipolar I Disorder—defined by manic episodes that last at least 7 days, or by manic symptoms that are so severe that the person needs immediate hospital care. Usually, depressive episodes occur as well, typically lasting at least 2 weeks. Episodes of depression with mixed features (having depression and manic symptoms at the same time) are also possible.

Bipolar II Disorder—defined by a pattern of depressive episodes and hypomanic episodes, but not the full-blown manic episodes described above.

As used herein ‘bipolar depression’ is defined as an individual who is experiencing depressive symptoms with a previous or coexisting episode of manic symptoms, but does not fit the clinical criteria for bipolar disorder.

As used herein, the term ‘anxiety disorder’ includes generalised anxiety disorder, phobia, panic disorder, social anxiety disorder, and post-traumatic stress disorder.

‘Generalised anxiety disorder’ (GAD) as used herein means a chronic disorder characterised by long-lasting anxiety that is not focused on any one object or situation. Those suffering from GAD experience non-specific persistent fear and worry, and become overly concerned with everyday matters. GAD is characterised by chronic excessive worry accompanied by three or more of the following symptoms: restlessness, fatigue, concentration problems, irritability, muscle tension, and sleep disturbance.

‘Phobia’ is defined as a persistent fear of an object or situation the affected person will go to great lengths to avoid, typically disproportional to the actual danger posed. If the feared object or situation cannot be avoided entirely, the affected person will endure it with marked distress and significant interference in social or occupational activities.

A patient suffering from a ‘panic disorder’ is defined as one who experiences one or more brief attack (also referred to as a panic attack) of intense terror and apprehension, often marked by trembling, shaking, confusion, dizziness, nausea, and/or difficulty breathing. A panic attack is defined as a fear or discomfort that abruptly arises and peaks in less than ten minutes.

‘Social anxiety disorder’ is defined as an intense fear and avoidance of negative public scrutiny, public embarrassment, humiliation, or social interaction. Social anxiety often manifests specific physical symptoms, including blushing, sweating, and difficulty speaking.

‘Post-traumatic stress disorder’ (PTSD) is an anxiety disorder that results from a traumatic experience. Post-traumatic stress can result from an extreme situation, such as combat, natural disaster, rape, hostage situations, child abuse, bullying, or even a serious accident. Common symptoms include hypervigilance, flashbacks, avoidant behaviours, anxiety, anger and depression.

As used herein, the term “post-partum depression” (PPD, also known as postnatal depression) is a form of depression experienced by either parent of a newborn baby. Symptoms typically develop within 4 weeks of delivery of the baby and often include extreme sadness, fatigue, anxiety, loss of interest or pleasure in hobbies and activities, irritability, and changes in sleeping or eating patterns.

As used herein, the term ‘substance abuse’ means a patterned use of a drug in which the user consumes the substance in amounts or with methods that are harmful to themselves or others.

As used herein, the term ‘an avolition disorder’ refers to a disorder that includes as a symptom the decrease in motivation to initiate and perform self-directed purposeful activities.

High-performance liquid chromatography (HPLC) is a technique in analytical chemistry used to separate, identify, and quantify each component in a mixture. For a review of HPLC, see A. M. Sabir et al., Int. Res. J. Pharm., 2013, 4, 4, 39-46.

As described above, the invention provides in its first aspect a parenteral formulation comprising a freebase of a deuterium-substituted DMT optionally substituted at position 4 or 5 with acetoxy or methoxy, and a biocompatible excipient.

By “parenteral formulation” is meant a formulation suitable for administration other than by the mouth or alimentary canal. The formulation (i.e. of the invention) is in accordance with Pharmacopeial requirements of sterility, contaminants, and pyrogens.

Sometimes, the formulation contains inhibitors of the growth of microorganisms (e.g. antimicrobial preservatives) and/or anti-oxidants.

Included within parenteral formulations are formulations suitable for inhalation or nasal, topical (including buccal, sublingual and transdermal), subcutaneous, intravenous or intramuscular administration.

In some embodiments, the parenteral formulation is suitable for inhalation, i.e. it is an inhalable formulation. For the avoidance of doubt, an inhalable formulation is capable of becoming airborne and entering the lungs of a patient through the action of the patient breathing in. In other words, inhalable formulations are suitable for pulmonary administration. The inhalable formulation may be inhaled in the form of a vapour, aerosol or gas. Often, the inhalable formulation is inhaled in the form of a vapour or aerosol.

The DMTs of use in connection with this invention are in the form of freebases. By “freebase” is meant that the amine within the DMT is in its unprotonated form, as opposed to the conjugate acid (protonated) form of the amine. Accordingly, salts of the DMTs are excluded from the scope of the freebase. For the avoidance of doubt, zwitterions comprising a protonated form of the amine and a negatively charged substituent bound to the DMT (such as the zwitterionic form of psilocybin) are excluded from the scope of the freebase.

The freebase is optionally substituted at position 4 or 5 with acetoxy or methoxy. For the avoidance of doubt, positions 4 and 5 of the optionally substituted DMT refer to the positions labelled in the structure below (substitution not shown).

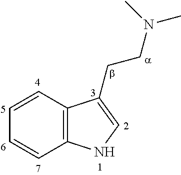

The DMT is substituted with deuterium, wherein a deuterium atom is a hydrogen atom with an additional neutron. For the avoidance of doubt, by a DMT (for example DMT itself or a substituted DMT such as 5-methoxy DMT) being substituted with deuterium is meant that such compounds are enriched with deuterium, i.e. they comprise a percentage of deuterium that is greater than the natural abundance of deuterium, which is about 0.015%. The deuterium of the deuterium-substituted DMT is typically bound to the alpha and/or beta positions of the compound (shown in the structure above). In particular embodiments, the carbon atom at the alpha position is bonded to at least one deuterium atom.

In some embodiments, the freebase is of Formula I

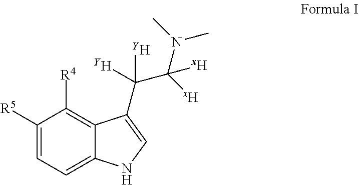

wherein:

one ^{x}H is D and the other is H or D; and

each ^{Y}H is independently selected from H and D; and

R⁴ and R⁵ are both H or one of R⁴ and R⁵ is H and the other is acetoxy (—OC(O)CH₃) or methoxy (—OCH₃).

In these embodiments, the compounds of Formula I comprise at least one deuterium atom at the α-position. In preferred embodiments, the compounds of Formula I comprise two hydrogen atoms at the β-position and at least one deuterium atom at the α-position. The inventors have found that such compounds are metabolised surprisingly slowly—substantially more slowly than their α-diprotic analogues—and consequently have improved parenteral (including inhalable) availability, allowing for a longer lasting therapeutic effect.

In some embodiments, both ^{x}H are D, i.e. the freebase is an α,α-dideutero compound.

In some embodiments, R⁴ and R⁵ are both H, i.e. the freebase is a deuterated analogue of DMT. In these embodiments, the deuterium-substituted DMT is any one of α-monodeutero-N,N-dimethyltryptamine, α,α-dideutero-N,N-dimethyltryptamine, α,β-dideutero-N,N-dimethyltryptamine, α,α,β-trideutero-N,N-dimethyltryptamine, α,β,β-trideutero-N,N-dimethyltryptamine and α,α,β,β-tetradeutero-N,N-dimethyltryptamine.

Herein, α,α-dideutero-N,N-dimethyltryptamines (such as α,α-dideutero-N,N-dimethyltryptamine or substituted α,α-dideutero-N,N-dimethyltryptamines, for example α,α-dideutero-5-methoxy-N,N-dimethyltryptamine) and α-protio, α-deutero-N,N-dimethyltryptamines (such as α-protio,α-deutero-N,N-dimethyltryptamine or substituted α-protio,α-deutero-N,N-dimethyltryptamines, for example α-protio,α-deutero-5-methoxy-N,N-dimethyltryptamine) are referred to as deuterated (or fully deuterated) N,N-dimethyltryptamines and partially deuterated N,N-dimethyltryptamines respectively. A deuterated (or fully deuterated) N,N-dimethyltryptamine thus refers strictly to an N,N-dimethyltryptamine with both protons at the α position substituted with deuterium atoms. The term partially deuterated N,N-dimethyltryptamine strictly refers to an N,N-dimethyltryptamine in which one of the two protons at the a position is substituted with a deuterium atom. A deuterated N,N-dimethyltryptamine herein is any N,N-dimethyltryptamine substituted with two deuterium atoms at the α position, and a partially deuterated N,N-dimethyltryptamine any N,N-dimethyltryptamine with one hydrogen atom and one deuterium atom at the α position.

Partially deuterated and deuterated DMTs can be synthesised following the reaction schemes (synthetic schemes) provided in Schemes 1 and 2 below. The chemistry depicted in the schemes was reported by P E Morris and C Chiao (Journal of Labelled Compounds And Radiopharmaceuticals, Vol. XXXIII, No. 6, 455-465 (1993)). Partially deuterated and deuterated DMT compounds can also be synthesised following the synthetic scheme depicted in Scheme 3.

If desired, formulations may comprise specific amounts of N,N-dimethyltryptamines (for example DMT itself or the R⁴- or R⁵-substituted DMTs described herein) and/or partially deuterated N,N-dimethyltryptamines, with the relative proportions of the N,N-dimethyltryptamine against the deuterated N,N-dimethyltryptamine and partially deuterated N,N-dimethyltryptamine controlled by varying the ratio of lithium aluminium hydride and lithium aluminium deuteride in the reducing agent. Relative proportions may further be varied by adding one or more of the N,N-dimethyltryptamine, α,α-dideutero-N,N-dimethyltryptamine and α,α,β,β-tetradeutero-N,N-dimethyltryptamine to the formulations described herein.

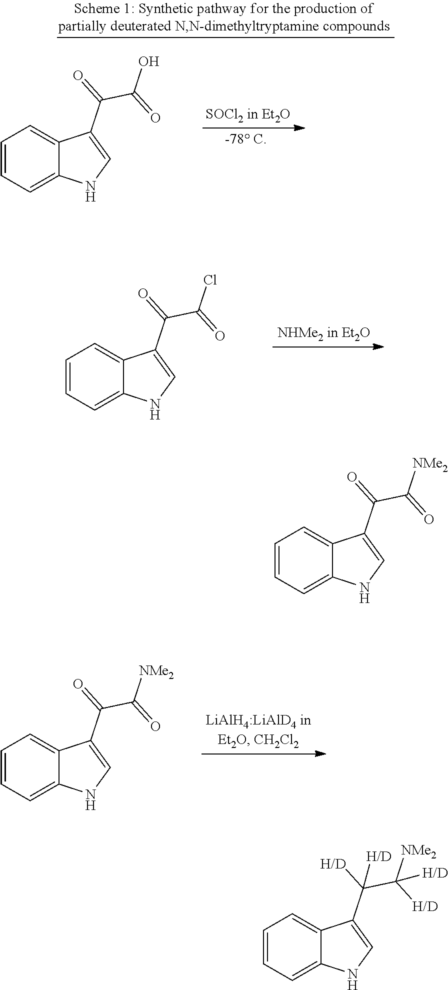

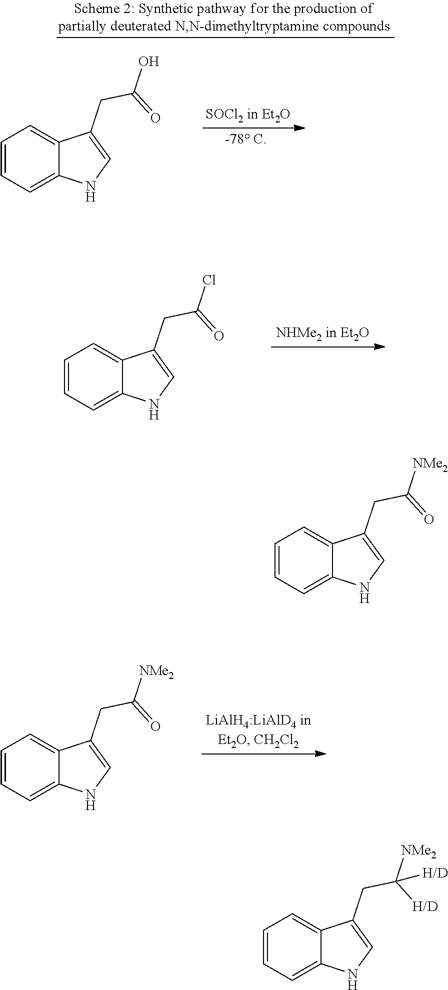

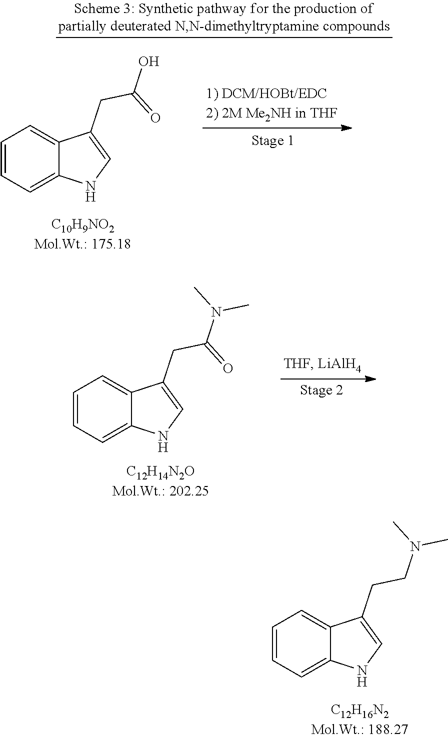

Identification of the compositions resultant from the reduction step in Schemes 1 and 2 may be achieved, if desired, by chromatographic separation of the components of the mixtures by conventional means at the disposal of the skilled person in combination with spectroscopic and/or mass spectrometric analysis.

Alternative compositions are obtainable by mixing N,N-dimethyltryptamine, obtainable by Scheme 1 or Scheme 2 when the reducing agent is exclusively lithium aluminium hydride, with a deuterated N,N-dimethyltryptamine compound obtainable from Scheme 1 or Scheme 2 when the reducing agent is exclusively lithium aluminium deuteride.

The compositions described hereinabove may be further modified by adding one or more deuterated or partially deuterated N,N-dimethyltryptamine compounds. Stocks of such deuterated or partially deuterated N,N-dimethyltryptamine compounds may be obtained, for example, from the chromatographic separation described above.

In some embodiments, R⁴ is acetoxy and R⁵ is H, i.e. the freebase (the deuterium-substituted DMT) is a deuterated analogue of O-Acetylpsilocin (4-AcO-DMT). In these embodiments, the freebase is any one of 4-acetoxy-α-monodeutero-N,N-dimethyltryptamine, 4-acetoxy-α,α-dideutero-N,N-dimethyltryptamine, 4-acetoxy-α,β-dideutero-N,N-dimethyltryptamine, 4-acetoxy-α,α,β-trideutero-N,N-dimethyltryptamine, 4-acetoxy-α,β,β-trideutero-N,N-dimethyltryptamine and 4-acetoxy-α,α,β,β-tetradeutero-N,N-dimethyltryptamine.

In particular embodiments, the freebase is 4-acetoxy-α-monodeutero-N,N-dimethyltryptamine or 4-acetoxy-α,α-dideutero-N,N-dimethyltryptamine.

In some embodiments, R⁴ is H and R⁵ is methoxy, i.e. the freebase (the deuterium-substituted DMT) is a deuterated analogue of 5-MeO-DMT. In these embodiments, the freebase is any one of 5-methoxy-α-monodeutero-N,N-dimethyltryptamine, 5-methoxy-α,α-dideutero-N,N-dimethyltryptamine, 5-methoxy-α,β-dideutero-N,N-dimethyltryptamine, 5-methoxy-α,α,β-trideutero-N,N-dimethyltryptamine, 5-methoxy-α,β,β-trideutero-N,N-dimethyltryptamine and 5-methoxy-α,α,β,β-tetradeutero-N,N-dimethyltryptamine.

In some embodiments, the freebase is 5-methoxy-α-monodeutero-N,N-dimethyltryptamine or 5-methoxy-α,α-dideutero-N,N-dimethyltryptamine.

Scheme 4 represents schemes known in the art to synthesise DMT compounds, in which substituent R¹ denotes hydrogen or the substituent R⁴ or R⁵, when other than hydrogen, as defined in Formula I, and each R² is methyl.

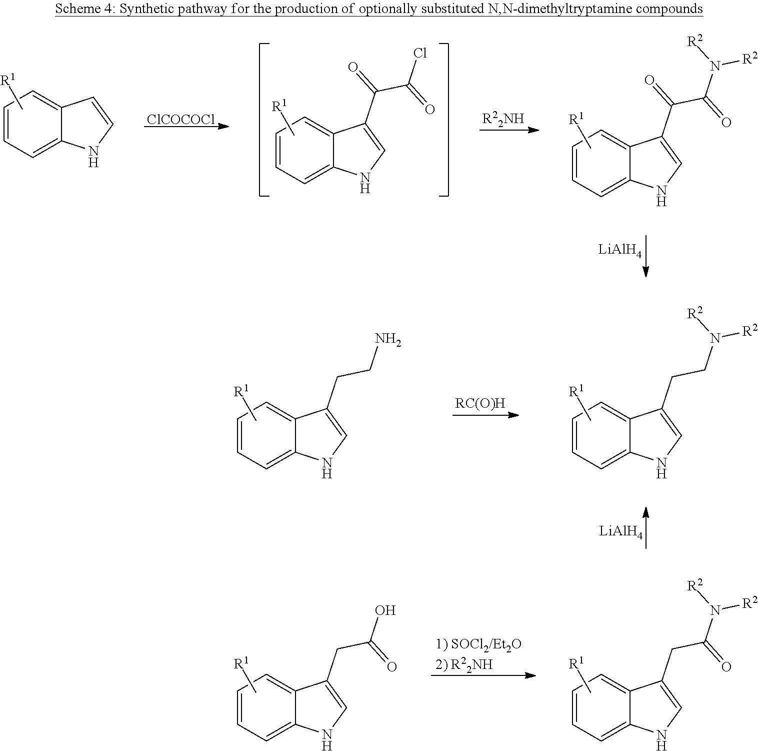

Mixtures of compounds of Formula I comprising controllable proportions of DMT and DMT with α-mono- and/or α,α-di-deuteration may, if desired, be prepared by reducing 2-(3-indolyl)-N,N-dimethyl acetamide with a desired ratio of lithium aluminium hydride and lithium aluminium deuteride. Similarly, mixtures comprising controllable proportions of DMT and DMT with α-mono-, α,α-di-deuteration, α,β-dideuteration, α,α,β-tri-deuteration, α,β,β-tri-deuteration and α,α,β,β-tetra-deuteration may, if desired, be prepared by reducing 2-(3-indolyl)-N,N-dimethyl-2-oxo-acetamide with a desired ratio of lithium aluminium hydride and lithium aluminium deuteride.

Analogous mixtures of compounds of Formula I comprising controllable proportions of R⁴ or R⁵-substituted DMT and R⁴ or R⁵-substituted DMT with α-mono-, α,α-di-deuteration, α,β-dideuteration, α,α,β-tri-deuteration, α,β,β-tri-deuteration and α,α,β,β-tetra-deuteration may be prepared by reducing R⁴ or R⁵-substituted 2-(3-indolyl)-N,N-dimethyl acetamide or R⁴ or R⁵-substituted 2-(3-indolyl)-N,N-dimethyl oxoacetamide with a desired ratio of lithium aluminium hydride and lithium aluminium deuteride.

In some cases, protecting groups may be useful. For example, to synthesise DMT substituted with acetyl, a benzyloxy 2-(3-indolyl)-N,N-dimethyl oxoacetamide may be reduced with a desired ratio of lithium aluminium hydride and lithium aluminium deuteride to produce a benzyloxy-N,N-dimethyl tryptamine (optionally substituted at the α and/or β positions with deuterium). The benzyl protecting group may then be removed, e.g. by hydrogenating with hydrogen and palladium on carbon, to form a hydroxy-N,N-dimethyl tryptamine (optionally substituted at the α and/or β positions with deuterium). The hydroxy group may be converted to an acetyl by reaction with acetic anhydride. See D. E. Nichols and S. Frescas, Synthesis, 1999, 6, 935-938 for further information on this synthetic strategy.

For more detail on the synthesis of DMT compounds, see the Example section herein.

Methods by which the compounds of Formula I may be produced are suitable for the production of high purity compounds of Formula I. The formulation may comprise a drug substance, which comprises the freebase of a deuterium-substituted dimethyltryptamine at a purity of greater than or equal to 99% when measured by HPLC. In some embodiments, the freebase is of a purity of greater than or equal to 99% by HPLC. Often, the freebase is of a purity of between 99% and 100% by HPLC, such as a purity of between 99.5% and 100% by HPLC. In some embodiments, the freebase, is of a purity of between 99.9% and 100% by HPLC, such as a purity of between 99.95% and 100% by HPLC.

The parenteral formulation comprises a biocompatible excipient. In particular embodiments, the biocompatible excipient comprises a solvent in which the freebase is at least partially soluble. The solvent is typically a liquid at ambient temperature and pressure (about 20° C. and about 1 bar). In more particular embodiments, the solvent is capable of forming a vapour or aerosol comprising the freebase on the application of heat, for example the solvent may be suitable for use in an electronic vaping device (EVD). EVDs typically include a power supply section and a cartridge. The power supply section often comprises a power source such as a battery, and the cartridge often comprises a heater and a reservoir capable of holding an inhalable formulation. The heater is typically contacted with the inhalable formulation (e.g. by a wick), and is typically configured to heat the inhalable formulation to generate a vapour or aerosol.

In some embodiments, the solvent is volatile (has a boiling point of ≤100° C., such as 50 to 100° C.). Such solvents may be capable of evaporation under the airflow of a vaporiser (such as a Volcano Medic Vaporizer) at temperatures of 30 to 70° C., e.g. 55° C. Evaporation of the solvent leaves a residue of freebase, which may then be vapourised into a vapour or aerosol under the airflow of a vapouriser at higher temperatures (e.g. at temperatures of about 150 to 250° C., such as 210° C.), and inhaled.

In some embodiments, the solvent is any one or a combination of two or more selected from the group consisting of propylene glycol (propane-1,2-diol), glycerine, polyethylene glycol, water, propanediol (propane-1,3-diol), butylene glycol (butane-1,3-diol), butane-2,3,-diol, butane-1,2-diol, ethanol and triacetin.

In some embodiments, the solvent is selected from propylene glycol, glycerine and polyethylene glycol, or a mixture thereof. Typically, the solvent is a mixture of propylene glycol and glycerine in a ratio of from about 50:50 (propylene glycol:glycerine) to about 10:90 by weight, such as about 50:50 to about 20:80 or about 50:50 to about 30:70 by weight. In some embodiments, the solvent is a mixture of propylene glycol and glycerine in a ratio of from about 50:50 to about 30:70 by weight.

Often, the glycerine is vegetable glycerine, i.e. glycerine derived from plant oils.

As described above, the formulations of the invention have uses in the treatment of psychiatric or neurological disorders. The concentration of the freebase within the formulation may be varied depending on the metabolic profile of the patient to whom the formulation is intended to be administered—lower concentrations may be more suitable for a patient with a slower metabolism. The inventors have found that compounds of Formula I comprising at least one deuterium atom at the α-position are metabolised substantially more slowly than their α-diprotic analogues and consequently exhibit long lasting therapeutic effects. Therefore, the concentration of freebase within the formulation may also be varied depending on the amount of deuterium within the freebase, particularly the amount of deuterium at the alpha and beta positions of the freebase—lower concentrations may be more suitable when using a freebase comprising a greater amount of deuterium. In some embodiments, the concentration of the freebase within the formulation is from about 1 mg/mL to about 1000 mg/mL, such as about 2 mg/mL to about 800 mg/mL or about 5 mg/mL to about 250 mg/mL.

Formulations suitable for inhalation or nasal administration often comprise a taste-masking agent. The purpose of the taste-masking agent is to make the taste or smell of the formulation more appealing to the patient. In some embodiments, the biocompatible excipient comprises a taste-masking agent. When the biocompatible excipient comprises a solvent and a taste-masking agent, the taste-masking agent is typically at least partially soluble in the solvent and the solvent is often able to form a vapour or aerosol comprising the freebase and the taste-masking agent on the application of heat. Often, the taste-masking agent is suitable for vaporisation into a vapour or aerosol under the airflow of a vapouriser (e.g. at temperatures of about 150 to 250° C., such as 210° C.). The taste-masking agent is typically a liquid or a solid at ambient temperature and pressure. It is advantageous that the taste-masking agent has no adverse effect on the bioavailability of the freebase, e.g. it is advantageous that the freebase is stable when stored in the presence of the taste-masking agent.

In some embodiments, the taste-masking agent is any one or a combination of two or more selected from the group consisting of flavourings, glucose, fructose, sorbitol, mannitol, honey, saccharin, sucrose, xylitol, erythritol, maltitol, sucralose, neotame, trehalose and tagatose. In some embodiments, flavourings are menthol, vanilla, wintergreen, peppermint, maple, apricot, peach, raspberry, walnut, butterscotch, wild cherry, chocolate, anise, citrus such as orange or lemon, or liquorice flavourings.

Examples of further biocompatible excipients that may be comprised within the parenteral formulation of the invention include but are not limited to those described in Gennaro et. al., Remmington: The Science and Practice of Pharmacy, 20^{th} Edition, Lippincott, Williams and Wilkins, 2000 (specifically part 5: pharmaceutical manufacturing). Suitable biocompatible excipients are also described in the Handbook of Pharmaceutical Excipients, 2^{nd} Edition; Editors A. Wade and P. J. Weller, American Pharmaceutical Association, Washington, The Pharmaceutical Press, London, 1994. M. F. Powell, T. Nguyen and L. Baloian provide a review of excipients suitable for parenteral administration in PDA J. Pharm. Sci. Technol., 52, 238-311 (1998). All soluble excipients listed in this review article are suitable excipients for use in the formulation.

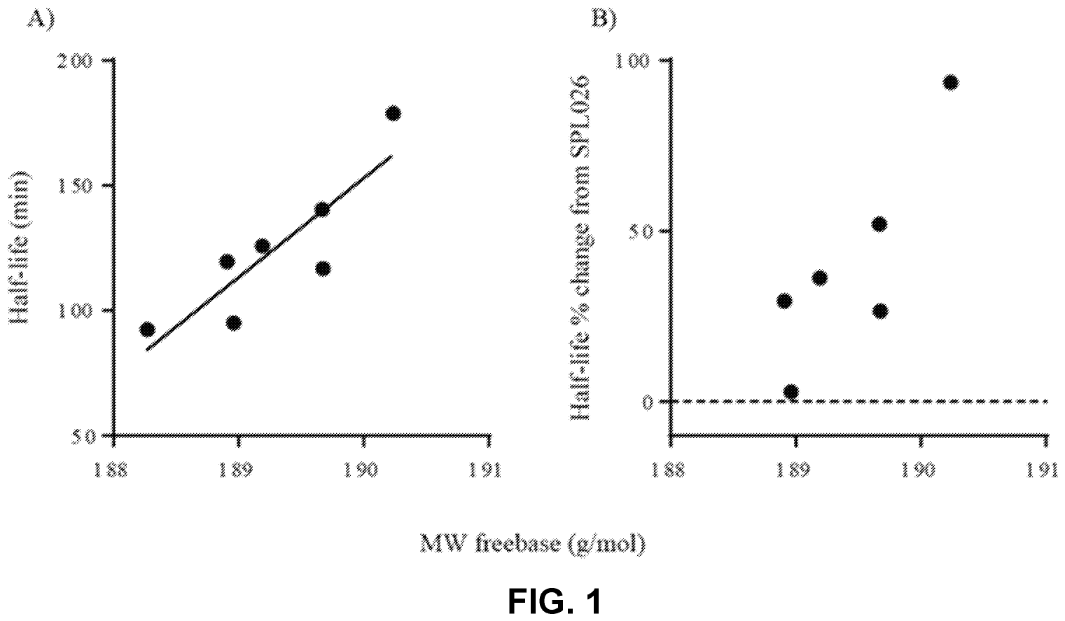

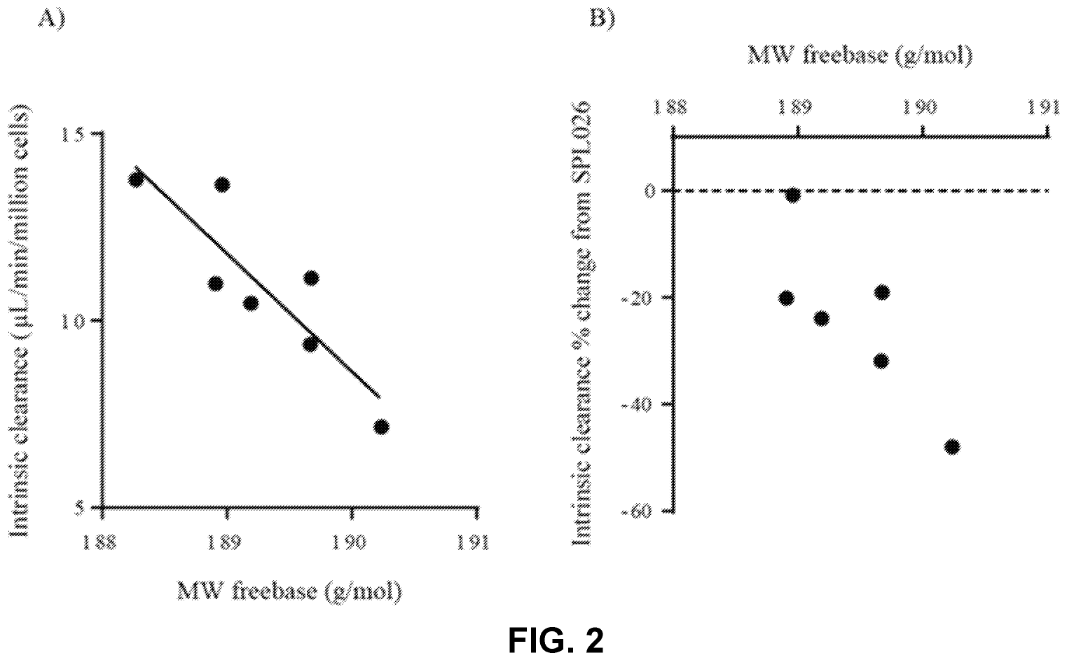

As detailed in the Examples section, and related FIGS. 1 and 2, the inventors have demonstrated that increasing deuterium enrichment at the α-carbon of N,N-dimethyltryptamine increases metabolic stability, leading to a decrease in clearance and longer half-life. A linear relationship exists between molecular weight and half-life, in particular when the input reducing agent for production of the deuterium-enriched N,N-dimethyltryptamine-containing compositions comprise LiAlH₄ and LiAlD₄ with a ratio between about 1:2.5 and about 2.5:1.

According to particular embodiments, the formulation of the first aspect comprises two freebases, a first freebase and a second freebase wherein the second freebase is an undeuterated analogue of the first freebase. In some embodiments, the mean molecular weight of the first freebase and second freebase is as defined in Table 1.

TABLE 1
Examples of formulations comprising two or more freebases

| | | Preferred m/w range |
|---|---|---|
| Compound | Substituent | of freebases in Daltons |
| DMT | — | 188.4-190.3 |
| 4-OAc-DMT | 4-OC(O)CH₃ | 246.4-248.3 |
| 5-OAc-DMT | 5-OC(O)CH₃ | 246.4-248.3 |
| 5-MeO-DMT | 5-OCH₃ | 218.4-220.3 |

In some embodiments, the formulation comprises three freebases, wherein the second freebase is an undeuterated analogue of the first freebase and the third freebase differs from the first freebase and the second freebase only by the number of deuterium atoms. In some embodiments, the mean molecular weight of the freebases is as defined in Table 1.

In some embodiments, the formulation consists essentially of a said first freebase, a said second freebase, optionally a said third freebase and one or more biocompatible excipients.

As used herein, mean molecular weight means the weighted average of molecular weights of the first freebase, second freebase and optional third freebase, as measured by an appropriate mass spectroscopic technique, for example LC-MS SIM (selected-ion monitoring). In some embodiments, the mean molecular weight is the weighted average.

It will be understood that providing compositions with such specific mean molecular weights can be achieved by those skilled in the art through the teachings herein, in particular by adjusting the relative proportions of lithium aluminium hydride and lithium aluminium deuteride in the reductions exemplified.

By reciting that the formulation consists essentially of the first freebase, second freebase, optional third freebase and one or more biocompatible excipients means that the formulation does not comprise material quantities of other pharmaceutically active compounds, including other dimethyltryptamine compounds.

In other words, and alternatively put, the formulations according to these specific embodiments constitute a drug substance comprising a biologically active ingredient consisting essentially of a mixture of the first freebase, second freebase and optional third freebase.

It will be understood that the formulation according to these specific embodiments comprises the first freebase and optional third freebase in amounts greater than found in isotopically unenriched protio analogues. It will also be understood that the greater the proportion of the first freebase and optional third freebase in these specific embodiments, the higher the mean molecular weight of the composition.

In some embodiments, the formulation has an oxygen content of less than 2 ppm, such as between 0.1 ppm and 2 ppm. The skilled person is able to determine the oxygen content of the formulation using any technique known in the art to be suitable, such as using a dissolved oxygen meter (e.g. a Jenway 970 Enterprise Dissolved Oxygen Meter, available from Keison Products: http://www.keison.co.uk/products/jenway/970.pdf.

The formulation may be stored in any suitable container. In some embodiments, to ameliorate degradation of the formulation, the formulation is stored in a container adapted to prevent penetration of ultraviolet light, such as an amber glass vial. In others, the container within which the formulation is stored is not so adapted (and may be, for example, made of clear glass) with protection against ultraviolet light, if desired, provided by secondary packaging (for example packaging within which the receptacle containing the formulation may be placed).

To ameliorate degradation of the formulation, it may be desirable to minimise the total oxygen content within the container in which the formulation is stored, the oxygen within the container equilibrating between the formulation and the headspace (if any) within the container. Accordingly, it may be desirable to store the formulation under an inert atmosphere for example by purging the headspace to reduce its oxygen content from about 20% typically found in air, to less than, for example, 0.5%. Often, the container is airtight and the formulation is stored under an inert atmosphere, such as under nitrogen or argon, typically nitrogen. The formulation may be stored at room temperature, e.g. at about 20 to about 30° C. or at cooler temperatures, for example at about 2 to about 8° C. Alternatively, to ameliorate degradation of the formulation further, it may be stored at temperatures lower than room temperature, such as in a refrigerator or freezer.

By means of pharmaceutically suitable liquids, the formulation can be prepared in the form of a solution, suspension, emulsion, or as a spray. Aqueous suspensions, isotonic saline solutions and sterile injectable solutions may be used, containing pharmaceutically acceptable dispersing agents and/or wetting agents, such as propylene glycol or butylene glycol.

The invention also provides a formulation of the invention, in combination with packaging material suitable for the formulation, the packaging material including instructions for the use of the formulation.

As described above, the invention provides in its second aspect a kit suitable for preparing a parenteral formulation of the first aspect, said kit comprising a freebase of a deuterium-substituted dimethyltryptamine optionally substituted at position 4 or 5 with acetoxy or methoxy, and a biocompatible excipient, which is separate to the freebase.

For the avoidance of doubt, embodiments related to the freebase and the biocompatible excipient of the first aspect of the invention as defined herein apply mutatis mutandis to the second aspect. For example, the freebase may be of Formula I, as defined above, and/or the biocompatible excipient may comprise a solvent selected from propylene glycol, glycerine and polyethylene glycol, or a mixture thereof.

The freebase within the kit may be a solid, e.g. in a powder or crystalline form. To ameliorate degradation of the freebase in the solid form, the salt may be lyophilised (freeze-dried) before incorporation into the kit. Lyophilising the salt comprises freezing it in the presence of solvent (typically water) and separating the solvent from the salt by sublimation.

In some embodiments, the kit further comprises a container adapted to prevent penetration of ultraviolet light, as described above. In such embodiments, the freebase within the kit is typically contained within the container.

As described above, the invention provides in its third aspect a method of preparing the parenteral formulation of the first aspect, comprising contacting a freebase of a deuterium-substituted dimethyltryptamine optionally substituted at position 4 or 5 with acetoxy or methoxy, with a biocompatible excipient.

For the avoidance of doubt, embodiments related to the freebase and the biocompatible excipient of the first aspect of the invention as defined herein apply mutatis mutandis to the third aspect. For example, the freebase may be of Formula I, as defined above, and/or the biocompatible excipient may comprise a solvent selected from propylene glycol, glycerine and polyethylene glycol, or a mixture thereof.

It will be understood that the contacting of the method may be achieved in a variety of ways. When the biocompatible excipient comprises a solvent, the freebase is often dissolved or suspended in the solvent. When the biocompatible excipient comprises a taste-masking agent, the freebase may be mixed with, dissolved in or suspended in the taste-masking agent. In cases where the biocompatible excipient comprises both a solvent and a taste-masking agent, the freebase and taste-masking agent may be dissolved or suspended in the solvent and may be added to the solvent in any order or simultaneously. Alternatively, the freebase may be mixed with, dissolved in or suspended in the taste-masking agent to form a first composition and the first composition may then be dissolved or suspended in the solvent.

In some embodiments, the method comprises dissolving the freebase in a solvent, such as a solvent selected from propylene glycol, glycerine and polyethylene glycol, or a mixture thereof, to form a solution. Typically, the solvent is a mixture of propylene glycol and glycerine in a ratio of from about 50:50 (propylene glycol:glycerine) to about 10:90 by weight, such as about 50:50 to about 20:80 or about 50:50 to about 30:70 by weight. In some embodiments, the solvent is a mixture of propylene glycol and glycerine in a ratio of from about 50:50 to about 30:70 by weight.

The skilled person is aware of techniques in the art suitable to promote dissolution of a solute such as the freebase into a solvent. Such techniques include agitating or stirring a composition comprising the solute and solvent, application of ultrasound, heating the composition and/or increasing the amount of solvent within the composition. When the method of the third aspect comprises dissolving the freebase in a solvent, the method often comprises stirring the freebase in the solvent, typically at a temperature of from about 25° C. to about 50° C.

Additionally or alternatively, in embodiments where the method comprises dissolving the freebase in a solvent to form a solution, the method may further comprise sparging the solution resultant from the contacting with an inert gas, such as nitrogen or argon, typically nitrogen.

In some embodiments, the method comprises adding a taste-masking agent. Sometimes, the method comprises dissolving the freebase in a solvent and then adding a taste-masking agent. The same techniques described above as suitable to encourage dissolution of a solute such as a freebase into a solvent also apply to the dissolution of a taste-masking agent into a solvent. Sometimes, the method comprises stirring the freebase and the taste-masking agent in the solvent, typically at a temperature of from about 25° C. to about 50° C.

The freebase within the formulation may be formed by contacting the analogous salt with a quantity of base suitable to deprotonate the conjugate acid (protonated) form of the amine. Accordingly, the method of the invention may comprise contacting a salt of a deuterium-substituted dimethyltryptamine optionally substituted at position 4 or 5 with acetoxy or methoxy, with a base and a biocompatible excipient. Sometimes, the base is any one selected from the group consisting of sodium hydroxide, potassium hydroxide, sodium carbonate, ammonium hydroxide, calcium hydroxide and magnesium hydroxide. In some embodiments, the base is adjusted with sodium hydroxide or potassium hydroxide.

As described above, the invention provides in its fourth aspect a parenteral formulation of the first aspect, or a kit of the second aspect for use in therapy.

For the avoidance of doubt, embodiments related to the parenteral formulation of the first aspect of the invention or the kit of the second aspect of the invention apply mutatis mutandis to the third aspect. For example, the formulation may be suitable for inhalation, the freebase may be of Formula I, as defined above, the biocompatible excipient may comprise a solvent selected from propylene glycol, glycerine and polyethylene glycol, or a mixture thereof, and/or the concentration of the freebase within the formulation may be from about 1 mg/mL to about 1000 mg/mL.

In some embodiments, the invention provides in its fourth aspect a parenteral formulation of the first aspect for use in therapy.

In some embodiments, the therapy is psychedelic-assisted psychotherapy, i.e. the therapy is treatment of a mental disorder by psychological means, which are enhanced by one or more protocols in which a patient is subjected to a psychedelic experience induced by administration of the formulation.

The invention provides in its fifth aspect a parenteral formulation of the first aspect, or a kit of the second aspect for use in a method of treating a psychiatric or neurological disorder in a patient.

In some embodiments, the invention provides in its fifth aspect a parenteral formulation of the first aspect for use in a method of treating a psychiatric or neurological disorder in a patient.

In another aspect, the invention provides use of a parenteral formulation of the first aspect, or a kit of the second aspect for the manufacture of a medicament. In some embodiments of this aspect, the medicament is for use in a method of treating a psychiatric or neurological disorder in a patient.

In some embodiments, the psychiatric or neurological disorder is selected from (i) an obsessive compulsive disorder, (ii) a depressive disorder, (iii) a schizophrenia disorder, (iv) a schizotypal disorder, (v) an anxiety disorder, (vi) substance abuse, and (vii) an avolition disorder. Often, the psychiatric or neurological disorder is selected from the group consisting of (i) an obsessive compulsive disorder, (ii) a depressive disorder, (iii) an anxiety disorder, (iv) substance abuse, and (v) an avolition disorder.

In some embodiments, the disorder is selected from the group consisting of major depressive disorder, treatment resistant major depressive disorder, post-partum depression, an obsessive compulsive disorder and an eating disorder such as a compulsive eating disorder.

In some embodiments, the psychiatric or neurological disorder is major depressive disorder. In some embodiments, the psychiatric or neurological disorder is treatment resistant depression.

As described above, the deuterium-substituted freebase within the formulation of the invention is metabolised more slowly than its protic analogue and consequently has improved parenteral (including inhalable) availability, allowing for a longer lasting therapeutic effect. Thus, in some embodiments, the therapy or method of treatment comprises parenteral administration, such as inhalation or pulmonary administration of the formulation.

Viewed from a sixth aspect, there is provided a method of treating a psychiatric or neurological disorder comprising pulmonary administration to a patient in need thereof of a parenteral formulation of the first aspect.

In some embodiments, the method of treatment is psychedelic-assisted psychotherapy, i.e. the method of treatment is treatment of a mental disorder by psychological means, which are enhanced by one or more protocols in which a patient is subjected to a psychedelic experience induced by administration of the formulation.

For the avoidance of doubt, embodiments related to the method of treatment, of the fifth aspect of the invention apply mutatis mutandis to the sixth aspect. For example, the disorder may be selected from the group consisting of (i) an obsessive compulsive disorder, (ii) a depressive disorder, (iii) an anxiety disorder, (iv) substance abuse, and (v) an avolition disorder.

In order to treat the disorder, an effective amount of the formulation is administered, i.e. an amount that is sufficient to reduce or halt the rate of progression of the disorder, or to ameliorate or cure the disorder and thus produce the desired therapeutic or inhibitory effect.

Each and every reference referred to herein is hereby incorporated by reference in its entirety, as if the entire content of each reference was set forth herein in its entirety.

The invention may be further understood with reference to the examples that follow.

### EXAMPLES

SPL028 (DMT, freebase) was prepared using the chemistry depicted in Scheme 5. SPL029 (5MeO-DMT, freebase) was prepared using the same procedure, but in which the indole-3-acetic acid starting material is replaced with 5-methoxyindole-3-acetic acid. An additional 4-6 g of six partially deuterated versions of SPL028 were also produced using modified conditions.

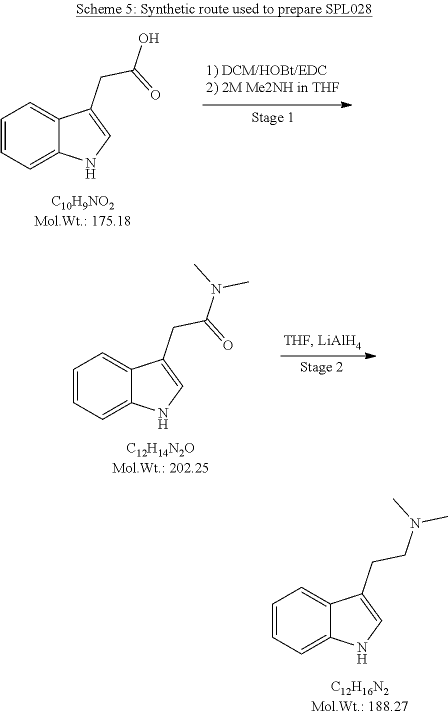

### SPL028 (DMT)

### Stage 1:coupling of indole-3-acetic acid and dimethylamine

To a 5 L vessel under N₂ was charged indole-3-acetic acid (257.0 g, 1.467 mol), Hydroxybenzotriazole (HOBt) (˜20% wet) (297.3 g, 1.760 mol) and dichloromethane (DCM) (2313 mL) to give a milky white suspension. Ethylcarbodiimide hydrochloride (EDC.HCl) (337.5 g, 1.760 mol) was then charged portion-wise over 5 minutes at 16-22° C. The reaction mixture was stirred for 2 hours at ambient temperature before 2 M dimethylamine in tetrahydrofuran (THF) (1100 mL, 2.200 mol) was charged dropwise over 20 minutes at 20-30° C. The resultant solution was stirred at ambient temperature for 1 hour where HPLC indicated 1.1% indole-3-acetic acid and 98.1% stage 1. The reaction mixture was then charged with 10% K₂CO₃ (1285 mL) and stirred for 5 minutes.

The layers were separated, and the upper aqueous layer extracted with DCM (643 mL×2). The organic extracts were combined and washed with saturated brine (643 mL). The organic extracts were then dried over MgSO₄, filtered and concentrated in vacuo at 45° C. This provided 303.1 g of crude stage 1 as an off-white sticky solid. The crude material was then subjected to a slurry in methyl-t-butyl ether (TBME) (2570 mL) at 50° C. for 2 hours before being cooled to ambient temperature, filtered and washed with TBME (514 mL×2). The filter-cake was then dried in vacuo at 50° C. to afford stage 1 266.2 g (yield=90%) as an off-white solid in a purity of 98.5% by HPLC and >95% by NMR.

### Stage 2: Preparation of DMT

To a 5 L vessel under N₂ was charged stage 1 (272.5 g, 1.347 mol) and THF (1363 mL) to give an off-white suspension. 2.4 M LiAlH₄ in THF (505.3 mL, 1.213 mol) was then charged dropwise over 35 minutes at 20-56° C. to give an amber solution. The solution was heated to 60° C. for 2 hours where HPLC indicated stage 1 ND, stage 2 92.5%, Impurity 1 2.6%, Impurity 2 1.9%. The complete reaction mixture was cooled to ambient temperature and then charged to a solution of 25% Rochelle's salts (aq.) (2725 mL) dropwise over 30 minutes at 20-30° C. The resultant milky white suspension was allowed to stir at 20-25° C. for 1 hour after which the layers were separated and the upper organic layer washed with saturated brine (681 mL). The organic layer was then dried over MgSO₄, filtered and concentrated in vacuo at 45° C. The resultant crude oil was subjected to an azeotrope from ethanol (545 mL×2). This provided 234.6 g (yield=92%) of stage 2 in a purity of 95.0% by HPLC and >95% by NMR.

### SPL029 (5MeO-DMT)

### Stage 1: Coupling of 5-Methoxyindole-3-Acetic Acid and Dimethylamine

To a 100 mL 3-neck flask under N₂ was charged 5-methoxyindole-3-acetic acid (3.978 g, 19.385 mmol), HOBt (˜20% wet) (3.927 g, 23.261 mmol) and DCM (40 mL). EDC.HCl (4.459 g, 23.261 mmol) was then charged in portions over 15 minutes at <30° C. The reaction mixture was stirred at ambient temperature for 1 hour before being charged with 2 M dimethylamine (14.54 mL, 29.078 mmol) dropwise over 15 minutes at <25° C. After stirring for 1 hour HPLC indicated no starting material (SM, i.e. 5-methoxyindole-3-acetic acid) remained. The reaction mixture was then charged with 10% K₂CO₃ (20 mL), stirred for 5 minutes then allowed to separate. The lower aqueous layer was removed and back extracted with DCM (10 mL×2). The organic extracts were combined, washed with saturated brine (10 mL) then dried over MgSO₄ and filtered. The filtrate was concentrated in vacuo at 45° C. to provide 3.898 g active (yield=87%) of product in a purity of 95.7% by HPLC.

### Stage 2: Preparation of 5MeO-DMT

To a 100 mL 3-neck flask under N₂ was charged stage 1 methoxy derivative (3.85 g, 16.586 mmol) and THF (19.25 mL). 2.4 M LiAlH₄ in THF (6.22 mL, 14.927 mmol) was then charged dropwise over 30 minutes at <40° C. The reaction mixture was heated to 60° C. for 1 hour where HPLC indicated 0.1% SM (stage 1 methoxy derivative) remained.

The reaction mixture was then cooled to ambient temperature and quenched into 25% Rochelle's salts (38.5 mL) dropwise over 30 minutes at <30° C. The resultant suspension was stirred for 1 hour before being allowed to separate. The lower aqueous layer was then removed, and the upper organic layer washed with saturated brine (9.6 mL). The organics were then dried over MgSO₄, filtered and concentrated in vacuo before being subjected to an azeotrope from EtOH (10 mL×2). This provided 3.167 g active (yield=88%) of product in a purity of 91.5% by HPLC.

### α,α-Dideutero-5-Methoxydimethyltryptamine

For stage 1 (coupling of 5-methoxyindole-3-acetic acid and dimethylamine), see above.

### Stage 2: Preparation of α,α-Dideutero-5-Methoxydimethyltryptamine

To a 100 mL 3-neck flask under N₂ was charged stage 1 methoxy derivative (3.85 g, 16.586 mmol) and THF (19.25 mL). 2.4 M LiAlD₄ in THF (6.22 mL, 14.927 mmol) was then charged dropwise over 30 minutes at <40° C. The reaction mixture was heated to 60° C. for 1 hour where HPLC indicated 0.1% SM (stage 1 methoxy derivative) remained. The reaction mixture was then cooled to ambient temperature and quenched into 25% Rochelle's salts (38.5 mL) dropwise over 30 minutes at <30° C. The resultant suspension was stirred for 1 hour before being allowed to separate. The lower aqueous layer was then removed, and the upper organic layer washed with saturated brine (9.6 mL). The organics were then dried over MgSO₄, filtered and concentrated in vacuo before being subjected to an azeotrope from EtOH (10 mL×2). This provided 3.196 g active (yield=88%) of product in a purity of 91.5% by HPLC.

### Synthesis of Deuterated Mixtures of DMT Compounds

A modified synthesis at stage 2 using solid LiAlH₄/LiAlD₄ mixtures was adopted, using 1.8 equivalents of LiAlH₄/LiAlD₄ versus 0.9 equivalents using the process described above for undeuterated DMT.

Six deuteration reactions were performed.

### Representative Synthesis of a Deuterated Mixture (Using 1:1 LiAlH₄:LiAlD₄) of DMT Compounds

To a 250 mL 3-neck flask under N2 was charged LiAlH₄ (1.013 g, 26.7 mmol), LiAlD₄ (1.120 g, 26.7 mmol) and THF (100 mL). The resultant suspension was stirred for 30 minutes before stage 1 (6 g, 29.666 mmol) was charged portion-wise over 15 minutes at 20-40° C. The reaction mixture was then heated to reflux (66° C.) for 2 hours where HPLC indicated no stage 1 remained. The mixture was cooled to 0° C. and quenched with 25% Rochelle's salts (aq) (120 mL) over 30 minutes at <30° C. The resultant milky suspension was stirred for 1 hour and then allowed to separate. The lower aqueous layer was removed and the upper organic layer washed with saturated brine (30 mL). The organics were then dried over MgSO₄, filtered and concentrated in vacuo. This provided 4.3 g of crude material. The crude was then taken up in ethanol (52 mL) and charged with fumaric acid (2.66 g, 22.917 mmol) before heating to 75° C. The resultant solution was allowed to cool to ambient temperature overnight before further cooling to 0-5° C. for 1 hour. The solids were isolated by filtration and washed with cold ethanol (6.5 mL×2). The filtercake was dried at 50° C. overnight to provided 5.7 g (yield=63%) of product in a purity of 99.9% by HPLC and >95% by NMR.

### Assessment of Extents of Deuteration

This was achieved by LCMS-SIM (SIM=single ion monitoring), the analysis giving a separate ion count for each mass for the three deuterated N,N-dimethyltryptamine compounds (N,N-dimethyltryptamine (D0), α-deutero-N,N-dimethyltryptamine (D1) and α,α-dideutero-N,N-dimethyltryptamine (D2)) at the retention time for N,N-dimethyltryptamine. The percentage of each component was then calculated from these ion counts.

For example, %D0=[D0/(D0+D1+D2)]×100.

| System: | Agilent 1100/1200 series liquid chromatograph | | | |
|---|---|---|---|---|
| | or equivalent | | | |
| Column: | Triart Phenyl; 150 × 4.6 mm, 3.0 μm particle size | | | |
| | (Ex: YMC, Part number: TPH12S03-1546PTH) | | | |
| Mobile phase A: | Water: Trifluoroacetic acid (100:0.05%) | | | |
| Mobile phase B: | Acetonitrile : Trifluoroacetic acid (100:0.05%) | | | |
| Gradient: | Time | | % A | % B |
| | 0 | | 95 | 5 |
| | 13 | | 62 | 38 |
| | 26 | | 5 | 95 |
| | 30.5 | | 5 | 95 |
| | 31 | | 95 | 5 |
| Flow rate: | 1.0 | mL/min | | |
| Stop time: | 31 | minutes | Post runtime: | 4 minutes |
| Injection volume: | 5 | μL | Wash vial: | N/A |

| | | | |
|---|---|---|---|
| Column temperature: | 30° C. combined | | |
| Wavelength: | 200 nm, (4 nm) | Reference: | N/A |

### Mass Spectrometry Parameters

| System: | Agilent 6100 series Quadrupole LC-MS or equivalent | | | |
|---|---|---|---|---|
| Drying gas flow: | 12.0 | L/min | Drying gas temp.: | 350° C. |
| Nebuliser pressure: | 35 | psig | | |

| | | | |
|---|---|---|---|
| Fragmentor: | 110 | Gain: | 1.00 |

| Cpd | RT | RRT | Conc | Diluent | Detection | Mass |
|---|---|---|---|---|---|---|
| D0 | 10.64 | 1.00 | 0.30 | CH₃CN:H₂O | (+) | 189.10 |
| | | | mg/ml | (50:50) | SIM | m/z |
| D1 | 10.64 | 1.00 | 0.30 | CH₃CN:H₂O | (+) | 190.10 |
| | | | mg/ml | (50:50) | SIM | m/z |
| D2 | 10.64 | 1.00 | 0.30 | CH₃CN:H₂O | (+) | 191.10 |
| | | | mg/ml | (50:50) | SIM | m/z |

MS-SIM range is the target mass ± 0.1 m/z

The data for the six deuterated reactions are tabulated in Table 2 below:

| Mixture | | | | | | | |
|---|---|---|---|---|---|---|---|
| No. | | | | | | | |
| (LiAlH₄: | Input | Output | Purity | Purity | | | |
| LiAlD₄ | (stage | stage 3 | by | by | Deuteration % | | |
| ratio) | 1) | (yield) | HPLC | NMR | D₀ | D₁ | D₂ |
| 1 | 5 g | 5.3 g | 99.7% | >95% | 0.7% | 2.7% | 96.6% |
| (SPL026) | | (65%) | | | | | |
| (0:1) | | | | | | | |
| 2 (1:1) | 6 g | 5.699 g | 99.9% | >95% | 30.0% | 48.3% | 21.7% |
| | | (63%) | | | | | |
| 3 (1:2) | 5 g | 4.206 g | 99.9% | >95% | 16.5% | 46.8% | 36.8% |
| | | (52%) | | | | | |
| 4 (1:3) | 5 g | 5.558 g | 99.8% | >95% | 9.3% | 41.5% | 49.2% |
| | | (68%) | | | | | |
| 5 (2:1) | 5 g | 4.218 g | 99.9% | >95% | 47.5% | 41.3% | 11.2% |
| | | (52%) | | | | | |
| 6 (3:1) | 5 g | 5.0 g | 99.4% | >95% | 57.5% | 35.3% | 7.4% |
| | | (62%) | | | | | |

### In Vitro Intrinsic Clearance of DMT (SPL026) and 6 Deuterated Compound Blends

In vitro determination of intrinsic clearance is a valuable model for predicting in vivo hepatic clearance. The liver is the main organ of drug metabolism in the body, containing both phase I and phase II drug metabolising enzymes, which are present in the intact cell.

### Aim

To use human hepatocytes to assess the in vitro intrinsic clearance of deuterated

DMT analogue blends relative to DMT.

### Description of the Experiment

Human (mixed gender) hepatocytes pooled from 10 donors (0.545 million cells/mL) were used to investigate the in vitro intrinsic clearance of DMT and 6 deuterated analogues.

A concentration of 5 μM was used for all test compounds, as well as sumatriptan, serotonin, benzylamine controls. This concentration was chosen in order to maximise the signal-to-noise ratio, while remaining under the Michaelis constant (Km) for the monoamine oxidase enzyme (MAO). Diltiazem and diclofenac controls were used at a laboratory-validated concentration of 1 μM.

Test compounds were mixed with the hepatocyte suspension within a 96-well plate and incubated for up to 60 minutes at 37° C. The suspension was continuously agitated. At 7 time points, small aliquots were withdrawn, and the test compound/blend concentration therein was measured by LC-MS/MS. The time points measured were 2, 4, 8, 15, 30, 45 and 60 minutes.

The following LC-MS/MS conditions were used for the analysis:
Instrument: Thermo TSQ Quantiva with Thermo Vanquish UPLC system
Column: Luna Omega 2.1×50 mm 2.6 μm
Solvent A: H₂O+0.1% formic acid
Solvent B: Acetonitrile+0.1% formic acid
Flow rate: 0.8 ml/min
Injection vol: 1 μl
Column temp: 65° C.
Gradient:

| Time (mins) | % Solvent B |
|---|---|
| 0.00 | 5.0 |
| 0.90 | 75.0 |
| 1.36 | 99.0 |
| 1.36 | 5.0 |
| 1.80 | 5.0 |

MS Parameters:

| | |
|---|---|
| Positive ion spray voltage: | 4000 V |
| Vaporiser temperature: | 450° C. |
| Ion transfer tube temp: | 365° C. |
| Sheath gas: | 54 |
| Aux gas: | 17 |
| Sweep gas: | 1 |
| Dwell time | 8 ms |
| MRM transitions: | D0 = mass to charge ratio 189.14 > 58.16. |
| | D1 = mass to charge ratio 190.14 > 59.17. |
| | D2 = mass to charge ratio 191.14 > 60.17. |

The MRM transitions were determined from a preliminary analysis of DMT samples containing either no deuterium (for D0 transition), or high levels of either D1 or D2 deuteration (for the D1 and D2 transitions respectively).

The resulting concentration-time profile was then used to calculate intrinsic clearance (CLint) and half-life (t½). To do this, the MS peak area or MS peak area/IS response of each analyte is plotted on a natural log scale on the y axis versus time (min) of sampling on the X axis. The slope of this line is the elimination rate constant. This is converted to a half-life by −In(2)/slope. Intrinsic clearance is calculated from the slope/elimination rate constant and the formula is CLint=(−1000*slope)/cell denisty in 1E6 cells/ml, to give units of microlitre/min/million cells.

### Results

Intrinsic clearance and half-life values were calculated for DMT and the 6 deuterated mixtures described above. These data were weighted dependent on the ratio of D0, D1 and D2 to give an overall intrinsic clearance and half-life value for each compound blend (Table 3).

TABLE 3
In vitro intrinsic clearance and calculated half-life of
DMT and 6 deuterated mixtures

| Compound name | | | |
|---|---|---|---|
| or Mixture No | LiAIH₄:LiAID₄ | D₀:D₁:D₂ | Half-life |
| (per Table 2) | input ratio | output ratio | (min) |
| DMT (SPL026) | 1:0 | 100:0:0 | 92.39 |
| 1 | 0:1 | 0.7:2.7:96.6 | 178.79 |
| 2 | 1:1 | 30.0:48.3:21.7 | 125.80 |
| 3 | 1:2 | 16.5:46.8:36.8 | 140.43 |
| 4 | 1:3 | 9.3:41.5:49.2 | 116.84 |
| 5 | 2:1 | 47.5:41.3:11.2 | 119.61 |
| 6 | 3:1 | 57.4:35.3:7.4 | 95.04 |

Data were fitted with a linear model using regression analysis, which revealed that deuterium enrichment at the α-carbon of DMT decreases intrinsic clearance linearly with increasing molecular weight (MW), therefore enabling manufacture of DMT drug substances with half-lives which can be accurately predicted in the range identified.

Mixture 1, which contains 96.6% D2-DMT, sees the biggest change, with the intrinsic clearance rate almost halved compared to undeuterated-DMT (FIG. 2), nearly doubling the half-life (FIG. 1). Intermediate blends of deuteration (Mixtures 2 to 5) decreased intrinsic clearance in a manner correlated with molecular weight (FIG. 2).

### Conclusion

These data demonstrate that increasing deuterium enrichment at the α-carbon of DMT increases metabolic stability, leading to a decrease in clearance and longer half-life. A linear relationship exists between MW and half-life, in particular when the input reducing agent for production of the deuterium enriched DMT-containing drug substance by methods of the present invention comprise LiAlH₄ and LiAlD₄ with ratio between 1:2.5 and 2.5:1. The relative half-life of analogous mixtures of protio, mono- and di-deutero compounds of substituted DMT such as compounds of Formula I are expected to mirror the trends observed here for mixtures of protio, mono- and di-deutero DMT. It is expected that increasing deuterium enrichment at the α-carbon of compounds of formula I increases metabolic stability, leading to a decrease in clearance and longer half-life.

### Inhalable Formulations of Deuterium-Enriched SPL028 and SPL029

Step 1: 1 g of either deuterium-enriched SPL028 freebase or deuterium enriched SPL029 freebase (referred to herein, for simplicity, as SPL029) is placed in a volumetric flask and dissolved in warm 100% ethanol to a concentration of 100 mg/ml.

Step 2: The resulting inhalable formulation may be varied in concentration to achieve a target dosage in the typical range of 1 mg to 25 mg SPL029 in 200 μl which is the dose volume delivered by a Volcano Medic Vaporizer. In accordance with the invention, for example, a 10 mg inhalable dose of SPL029 is delivered by diluting the stock solution 1:1 with 100% ethanol SPL029, providing 50 mg of SPL029 dissolved in 100% ethanol for a final solution volume of 1 ml. Aliquots of the stock solution are stored in vials until further use.

Step 3: 200 ul of the 50 mg/ml solution is transferred to a dosing capsule containing a drip pad (Storz & Bickel, Germany).

Step 4: The SPL029 charged dosing capsule is transferred to the filling chamber of a preheated Volcano Medic Vaporizer (55° C.). The airflow of the vaporizer is switched on for 60 seconds at a pre-set rate of 12 l/min to evaporate the ethanol solvent. The 10 mg dose of SPL029 is confirmed by subtracting the weight of the dosing capsule plus residue from the empty weight of the dosing capsule.

Step 5: The prepared dosing capsule is returned to the filling chamber of the Volcano Medic Vaporizer, now pre-heated to 210° C., with the airflow on for at least 5 minutes immediately prior to transfer. An inhalation balloon with valve attached (Storz & Bickel, Germany) is mounted on the socket of the filling chamber. Once closed and sealed, the airflow is switched on for 15 seconds at a flow rate of 12 l/min, allowing the full dose of SPL029 to aerosolize within the balloon in a volume of about 3 litres.

Step 6: The balloon is disconnected from the filling chamber, automatically closing the valve. The aerosolized dose of SPL029 can then be administered to a patient via the balloon.

Step 7: To prepare for the administration, a patient is advised to perform several deep inhalations and exhalations, in order to empty the lungs. Then, with the mouthpiece firmly held against the lips, the full and complete volume of the inhalation balloon is inhaled in one inhalation, holding the breath for about 10 seconds, followed by a normal exhalation. Repeated inhalations into and out of the balloon can aid in maximum absorption of the SPL029 dose by the patient.

### Alternative Inhalable Formulations of Deuterium-Enriched SPL028 and SPL029

An alternative way to prepare inhalable formulations in accordance with the invention is to prepare a liquid solution of either deuterium-enriched SPL028 freebase or deuterium-enriched SPL029 freebase which is compatible with an electric vaporizer, e.g. A SMOK AL85 or Alike 40W (available on Amazon). Generally, any electric vaporizer with output current of 20 Amps or more will be compatible with the formulations described in this example.

Step 1: To a 4 ml glass vial which is compatible with an electric vaporizer (e.g. A sub ohm tank), add 3 ml of vegetable glycerine (or any premixed combination of propylene glycol and vegetable glycerine, also known as ‘vape juice’), and then add 0.75 g of either deuterium-enriched SPL028 freebase or deuterium enriched SPL029 free base (referred to herein, for simplicity, as SPL028). The SPL028 will dissolve when left for a few hours, and will dissolve more quickly with gentle heating in a water bath. If any material remains undissolved, extract the solution with a syringe fitted with a filter and transfer the filtrate to a clean vial.

Step 2: Attach and secure the vial to the vaporizer and switch on the power. The patient then uses the vaporiser according to the instructions provided. When this method is used, the patient can self-titrate to an effective dose, usually under the guidance of a guide or a healthcare professional. Advantages of this invention over DMT vapes known in the art include a reduction of the number and frequency of inhalations necessary to achieve a particular psychedelic experience. Moreover, the extended half-life provides for an extended and/or tailored duration of experience.

1. A parenteral formulation comprising a freebase of a deuterium-substituted dimethyltryptamine optionally substituted at position 4 or 5 with acetoxy or methoxy, and a biocompatible excipient.

2. The formulation of claim 1, wherein the formulation is suitable for inhalation.

3. The formulation of claim 1, wherein the freebase is of Formula I 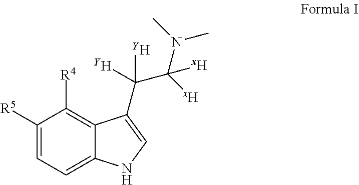
wherein:
one ^{x}H is D and the other is H or D;
each ^{Y}H is independently selected from H and D; and
R⁴ and R⁵ are both H or one of R⁴ and R⁵ is H and the other is acetoxy (—OC(O)CH₃) or methoxy (—OCH₃).

4. The formulation of claim 3 wherein both ^{Y}H are H.

5. The formulation of claim 3 wherein R⁴ and R⁵ are both H.

6. The formulation of claim 3 wherein R⁴ is acetoxy and R⁵ is H.

7. The formulation of claim 3 wherein R⁴ is H and R⁵ is methoxy.

8. The formulation of claim 1 wherein the freebase has a purity of greater than or equal to 99% when measured by HPLC.

9.-33 (canceled)

34. The formulation of claim 1 further comprising a solvent comprising propylene glycol, glycerine and polyethylene glycol, or a mixture thereof.

35. The formulation of claim 34, wherein the solvent is a mixture of propylene glycol and glycerine in a ratio of from about 50:50 to about 30:70 by weight.

36. The formulation of claim 1 further comprising a taste-masking agent.

37. The formulation of claim 1 wherein the formulation has an oxygen content of less than 2 ppm.

38. A kit suitable for preparing a parenteral formulation of claim 3, said kit comprising:
a container adapted to prevent penetration of ultraviolet light.

39. A method of preparing a parenteral formulation of claim 1, comprising contacting a freebase of a deuterium-substituted dimethyltryptamine optionally substituted at position 4 or 5 with acetoxy or methoxy, with a biocompatible excipient.

40. The method of claim 39 wherein the freebase is dissolved in a solvent selected from propylene glycol, glycerine and polyethylene glycol, or a mixture thereof to form a solution.

41. The method of claim 40 wherein the solvent is a mixture of propylene glycol and glycerine in a ratio of from about 50:50 to about 30:70 by weight.

42. The method of claim 40 further comprising stirring the freebase in the solvent at a temperature of from about 25° C. to about 50° C.

43. The method of claim 40 further comprising sparging the solution resultant from the contacting with an inert gas.

44. A method of treating a psychiatric or neurological disorder comprising pulmonary administration to a patient in need thereof of the parenteral formulation of claim 1.

45. The method of claim 44 wherein the psychiatric or neurological disorder is selected from the group consisting of (i) an obsessive compulsive disorder, (ii) a depressive disorder, (iii) an anxiety disorder, (iv) substance abuse, and (v) an avolition disorder.
